(19) United States
(12) Patent Application Publication
(10) Pub. No.: US 2007/0055077 A1
(43) Pub. Date: Mar. 8, 2007

(54) CHEMICAL PROCESS

(75) Inventors: Stephen Martin Brown, Huddersfield, West Yorkshire (GB); James Peter Muxworthy, Huddersfield, West Yorkshire (GB)

Correspondence Address: SYNGENTA CROP PROTECTION , INC.;PATENT AND TRADEMARK DEPARTMENT, 410 SWING ROAD, GREENSBORO, NC, 27409, US

(73) Assignee: SYNGENTA LIMITED, Guildford (GB)

(21) Appl. No.: 11/554,076

(22) Filed: Oct. 30, 2006

Related U.S. Application Data
(63) Continuation of application No. 10/260,024, filed on May 16, 2003, now abandoned.
Application No. 10/260,024 is a continuation of application No. 08/712,695, filed on Sep. 11, 1996, now abandoned.

(30) Foreign Application Priority Data
Sep. 13, 1995 (GB) 9518705.0

(51) Int. Cl. C07C 313/26

(52) U.S. Cl. 562/435; 564/87

(57) ABSTRACT

A process for the preparation of a compound of general formula I: 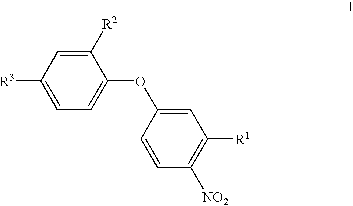 wherein:
R¹ is hydrogen or C₁-C₆ alkyl, C₂-C₆ alkenyl or C₂-C₆ alkynyl (any of which may optionally be substituted with one or more substituents selected from halogen and OH) or COOH, COH, COOR , COR⁶, CONR⁴R⁵ or CONHSO₂R⁴;
R⁴ and R⁵ are each independently hydrogen or C₁-C₄ alkyl optionally substituted with one or more halogen atoms;
R⁶ is a halogen atom or a group R⁴;
   R² is hydrogen or halo;
   R³ is C₁-C₄ alkyl, C₂-C₄ alkenyl or C₂-C₄ alkynyl, any of which may optionally be substituted with one or more halogen atoms, or halo;
   the process comprising reacting a compound of general formula II: 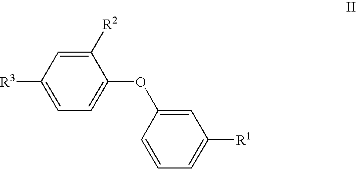
   wherein R¹, R² and R³ are as defined for general formula I; with a nitrating agent comprising nitric acid or a mixture of nitric and sulphuric acids in the presence of an organic solvent and in the presence of acetic anhydride, characterised in that the molar ratio of acetic anhydride to compound of general formula I is from about 1:1 to 3:1.

This application is a continuation of U.S. application Ser. No. 10/260,024 filed May 16, 2003, still pending, which is a continuation of U.S. application Ser. No. 08/712,695, filed Sep. 11, 1996, now abandoned, which claims the benefit of GB Application No. 9518705.0, filed Sep. 13, 1995, now abandoned, the contents of which are incorporated herein by reference.

The present invention relates to a process for nitration and, in particular to a process for nitrating diphenyl ether compounds which are useful as herbicides or as intermediates in the synthesis of herbicides.

EP-A-0022610 relates to herbicides of the formula: 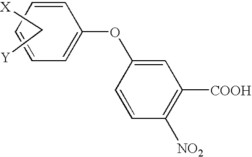
wherein X and Y may be H, F Cl, Br, CF₃, OCF₂CHZ₂ (Z=Cl, Br, F), OCH₃, CN, CO₂R (R=lower alkyl), C₆H₅, O-alkyl, NO₂ or SO₂ lower alkyl;
and also describes a process for making these compounds by nitrating a compound of the formula: 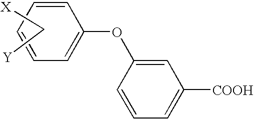
wherein X and Y are as defined above.

Suggested nitrating agents for this reaction include mixtures of nitric and sulphuric acids and the recommended reaction solvent is dichloromethane. The nitration process is said to give a yield of 75.4% but no details are given of the purity of the product or the presence of other nitrated isomers.

U.S. Pat. No. 4,031,131 describes similar compounds to the above which are prepared in a similar manner. Suggested nitrating agents include potassium nitrate or mixed nitric and sulphuric acids and the reaction is carried out in dichloromethane. An extremely high yield (>95%) is claimed for the nitration reaction but, again, there are no details given about the purity of the product. Nitration reactions using mixed nitric and sulphuric acids may also be carried out in the presence of acetic anhydride.

EP-A-0003416 and EP-A-0274194 both relate to the synthesis of herbicidal compounds of the formula: 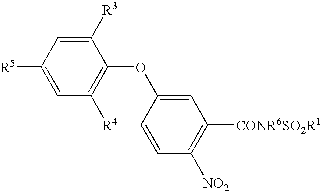
wherein R¹ is alkyl optionally substituted with fluorine or optionally substituted phenyl;
R³ is H, F, Cl, Br, I alkyl, trifluoromethyl or CN;
R⁴ is H, F, Cl, Br, I or trifluoromethyl;
R⁵ is F, Cl, Br, I or trifluoromethyl; and
R⁶ is H or C₁-C₄ alkyl.

In EP-A-0003416, these compounds may be obtained by nitrating the corresponding carboxylic acid or carboxamide and then converting to the sulphonamide or by nitrating the sulphonamide itself. A nitration reaction is described in Example 7 where the solvent is 1,2-dichloroethane and the nitrating agent is a mixture of potassium nitrate and concentrated sulphuric acid.

EP-A-0274194 relates, in particular, to a process for the nitration of compounds of the formula:

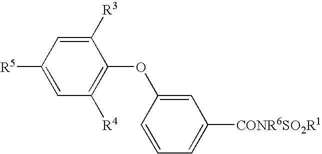

The nitration reaction is said to be carried out using a conventional nitrating agent such as concentrated nitric acid or sodium nitrate or mixtures of these with sulphuric acid. The reaction solvent is one which is resistant to nitration and examples of such solvents are said to include halogenated solvents such as dichloromethane, dichloroethane, dichloropropane, chlorofluorocarbons and aromatic solvents such as nitrobenzene.

However, none of these methods are particularly satisfactory for use on an industrial scale because they all have the common problem that the reaction yields a mixture of the required product and other nitrated isomers. Nitrated isomers of diphenyl ether compounds are often extremely difficult to separate from one another and the quantity of other isomers is often too high for the final product to fulfill the requirements of the regulatory authorities for herbicides. The problem tends to be further exacerbated if the nitrated product is an intermediate in the synthesis of a herbicide rather than the required herbicide itself, because the mixture of nitrated compounds means that larger quantities of other reagents must be used than would be necessary if the nitrated isomers could be separated satisfactorily. It is therefore important to ensure that the nitration process produces a product mixture containing the highest possible proportion of the desired isomer.

The problem of obtaining mixtures of isomers from the nitration process was recognised by the authors of GB-A-2103214 who describe a process in which a compound of the formula: 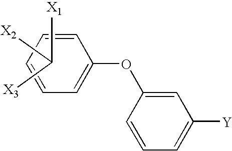
wherein each of X₁, X₂, and X₃, is H, fluorine, chlorine, bromine, CF₃, O CF₂,CHZ₂ (where Z is F, Cl or Br), OCF₃, CN, COOR (R is lower alkyl), phenyl, lower alkoxy or NO₂R and at least one of X₁, X₂, and X₃ is other than hydrogen; and
Y is COOR or carboxy;
is nitrated to give a product of the formula: 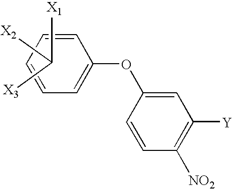
wherein X₁, X₂, X₃ and Y are as defined above.

The nitration is carried out using as nitrating agent a mixture of nitric and sulphuric acids in an organic solvent such as dichloromethane. The desirability of keeping the reaction system anhydrous by the addition of acetic anhydride is stressed as the authors of GB-A-2103214 state that this makes it possible to improve the selectivity with respect to Acifluorfen (the desired nitrated product). The recommended ratio of starting material: solvent : acetic anhydride is 1:2.66:1.4. The reaction is conducted at a temperature of 45° C. and left for 3 hours. After this, the reaction mixture is allowed to stand so that the organic and aqueous phases separate and then the organic solvent is removed by distillation.

However, the present inventors have found that the use of reaction conditions suggested lead to various problems which do not seem to have been appreciated by the authors of the prior art document. In particular, although the use of acetic anhydride does, in some respects, improve the selectivity of the reaction, the relationship between the concentration of acetic anhydride and selectivity is more complex than the authors of GB-A-2103214 appear to have realised and, therefore, the amount of acetic anhydride in the reaction mixture must be carefully controlled in order to obtain a suitable product mixture.

Therefore in the present invention there is provided a process for the preparation of a compound of general formula I: 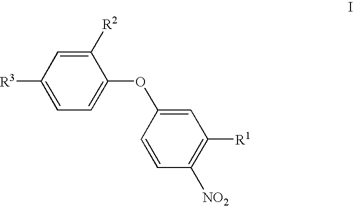
wherein:
R¹ is hydrogen or C₁-C₆ alkyl, C₂-C₆ alkenyl or C₂-C₆ alkynyl (any of which may optionally be substituted with one or more substituents selected from halogen and OH) or COOH, COH, COOR⁴,COR⁶, CONR⁴R⁵ or CONHSO₂R⁴;
R⁴ and R⁵ are each independently hydrogen or C₁-C₄ alkyl optionally substituted with one or more halogen atoms;
R⁶ is a halogen atom or a group R⁴;
   R² is hydrogen or halo;
   R³ is C₁-C₄ alkyl, C₂-C₄ alkenyl or C₂-C₄ alkynyl, any of which may optionally be substituted with one or more halogen atoms, or halo;
   the process comprising reacting a compound of general formula II: 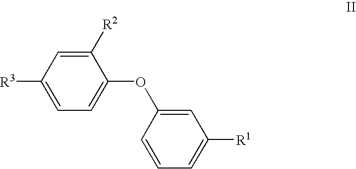
   wherein R¹, R² and R³ are as defined for general formula I;
   with a nitrating agent comprising nitric acid or a mixture of nitric and sulphuric acids in the presence of an organic solvent and in the presence of acetic anhydride, characterised in that the molar ratio of acetic anhydride to compound of general formula II is from about 1:1 to 3:1.

These reaction conditions give the advantage that the proportion of the required isomer is maximised whilst not causing too great a reduction in the yield of the product or too great an increase in operating costs.

In the context of the present invention, compounds of general formula I are designated 4-nitro isomers. The 2-nitro isomers referred to above have the general formula: 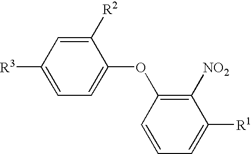
Other mono-nitro isomers which may be produced in the nitration reaction include the 6-nitro isomer:

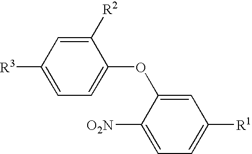

There are also three different dinitro isomers which may be present.

In the context of the present invention, the term “C₁-C₆ alkyl” refers to a saturated straight or branched hydrocarbon chain containing from 1 to 6 carbon atoms. Examples include methyl, ethyl, n-propyl, t-butyl, n-pentyl and n-hexyl. The term “C₁-C₄ alkyl” is a subset of C₁-C₆ alkyl and refers to an alkyl group having up to 4 carbon atoms.

The term “C₂-C₆ alkenyl” refers to a straight or branched hydrocarbon chain containing from 2 to 6 carbon atoms and having at least one double bond. Examples include ethenyl, allyl, propenyl and hexenyl. The term “C₂-C₄ alkenyl” is a subset of C₂-C₆ alkenyl and refers to an alkenyl group having up to 4 carbon atoms.

The term “C₂-C₆ alkynyl” refers to a straight or branched hydrocarbon chain containing from 2 to 6 carbon atoms and having at least one triple bond. Examples include ethynyl, propynyl and hexynyl. The term “C₂-C₄ alkynyl” is a subset of C₂-C₆ alkynyl and refers to an alkynyl group having up to 4 carbon atoms.

The term “halogen” refers to fluorine, chlorine, bromine or iodine and the corresponding term “halo” refers to fluoro, chloro, bromo or iodo.

The reaction conditions of the present invention are particularly advantageous since they maximise the amount of the required 4-nitro isomer in the product mixture. Surprisingly, it has been found by the present inventors that the relationship between the presence of acetic anhydride and the isomer ratio of the product mixture is not as simple as it appears from a reading of GB-A-2103214. This document suggests that the presence of acetic anhydride is beneficial but does not suggest that the amount present needs to be limited. The present inventors have found, however, that although the proportion of dinitro isomers (1) and (2) in the product mixture decreases as the amount of acetic anhydride is increased, the proportion of the 2-nitro impurity increases. This is a particular concern since the 2-nitro isomer is especially difficult to separate from the 4-nitro isomer and so, clearly, it is important to keep its concentration in the product mixture as low as possible. For this reason, the present inventors have found that it is not desirable to increase the acetic anhydride: compound II ratio to greater than about 3:1.

Additionally, the present inventors have discovered that the reaction temperature plays a significant role in determining the proportions of the various mono-nitrated isomers with a greater proportion of the required isomer being produced as the reaction temperature is reduced. The reaction temperature, too is a compromise since, clearly, it would not be economically viable to operate a reaction if the temperature were below a certain level because of the amount of cooling required. The decrease with temperature of the proportion of the 2-nitro and 6-nitro isomers in the product mixture does not seem to have been appreciated by the authors of GB-A-2103214 who recommended a reaction temperature of about 45° C. The present inventors have found that the amount of the 2-nitro isomer present in the product mixture when the reaction temperature is 45° C. is more than 12 parts per hundred whereas, when the reaction temperature is reduced to 10° C., the amount of 2-nitro isomer in the product mixture is reduced to 10 or 11 parts per hundred. This difference may affect any subsequent purification process and may be very significant when costing a large scale manufacturing process. The preferred temperature range for the process of the present invention is from about −15° to 15° C., more preferably −10° to 10° C.

It has also been found that the formation of the undesired isomers can be further reduced by increasing the concentration of the reactants in the solvent solution. In particular, it is advantageous to have a weight ratio of solvent to reactant (including any isomers present) of no greater than 4.25:1 and it is preferred that the ratio is from 1:1 to 2.5:1.

The reaction may be carried out in any suitable solvent and examples of solvents which may be used include halogenated solvents such as dichloromethane (DCM), ethylene dichloride (EDC), chloroform, tetrachloroethylene (perklone) and dichlorobenzotrifluoride (DCBTF). Alternatively, solvents such as acetic acid, acetonitrile, ethers such as tetrahydrofuran (THF) or dioxane, sulpholane, nitrobenzene, nitromethane, liquid sulphur dioxide or liquid carbon dioxide may all be used successfully in the reaction.

Perklone is a particularly useful solvent for the process of the present invention since, under equivalent reaction conditions, Perklone reactions give about 30% less of the 2- and 6-nitro isomers than reactions carried out in EDC or DCM under otherwise identical conditions. There are also indications that the yield of the reaction is increased when Perklone is the solvent of choice.

As already mentioned, the nitrating agent used is nitric acid or a mixture of nitric and sulphuric acids. A mixture of nitric and sulphuric acids may contain, for example, from about 30 to 45% of pure nitric acid, more typically from about 30 to 35% pure nitric acid.

When the chosen nitrating agent is a mixed acid, it will typically be added to the reaction mixture over a period of about 30 minutes to 15 hours. The rate of addition will, however vary according to the reaction solvent which is chosen with addition over about 1 to 6 hours, or preferably 2 to 4 hours, being appropriate for many solvents, for example EDC and DCM.

When the reaction is conducted in Perklone, however, the rate of reaction is usually somewhat lower than for reactions conducted in other solvents such as EDC or DCM and so it is often advantageous to add the nitrating agent more slowly, for example over a period of from 5 to 15 hours, or, more preferably, 6 to 12 hours.

Although the process of the invention may be used for the preparation of any compound of general formula I, it is especially preferred that R² is chloro and R³ is trifluoromethyl. Particularly preferred compounds of general formula I are those in which R¹ is COOH or CONHSO₂CH₃. These compounds are 5-(2-chloro-α,α,α-trifluoro-4-tolyloxy)-2-nitrobenzoic acid (Acifluorfen) and 5-(2-chloro-α,α,α-trifluoro-4-tolyloxy)-N-methanesulphonyl-2-nitrobenzamide (Fomesafen), both of which are potent herbicides.

In addition to being a herbicide in its own right, Acifluorfen may also serve as an intermediate in the synthesis of Fomesafen. The Acifluorfen may be converted to the acid chloride which may then be reacted with methane sulphonamide to give Fomesafen. Both of these steps may be carried out by conventional methods, for example as set out in EP-A-0003416.

The invention will now be further described by way of the following examples in which the following abbreviations are used:

DCM—dichloromethane;

EDC—ethylene dichloride

pph—parts per hundred;

HPLC—high performance liquid chromatography.

In the examples, the term “mixed acid” refers to a mixture containing 33.6% nitric acid and 66.4% sulphuric acid. The molar quantities given are the moles of nitric acid in the mixture.

### EXAMPLE 1

### General Method for

Nitration of 3-(2-chloro-α,α,α-trifluoro-4-tolyloxy)benzoic acid in Dichloromethane to Yield Acifluorfen

Nitration

Acetic anhydride (see Tables I and II for amounts) was added to 3-(2-chloro-α,α,α-trifluoro-4-tolyloxy)benzoic acid (I, R¹ is COOH, R² is chloro, R³ is trifluoromethyl) (20 g, 0.063 mol) in dichloromethane (54 g, 0.635 mol) and the mixture stirred and heated to 40° C. to dissolve the starting material. The mixture was then cooled to the appropriate reaction temperature (during which time any crystallisation of the starting material was observed). Mixed acid (13 g, 0.069 mol) was added dropwise over a period of 2 hours and the reaction monitored by HPLC for the completion of the reaction. Further additions of Mixed acid were made to reduce the level of starting material to about 1 pph.

Work-Up

The reaction mixture was washed three times as follows:

wash 1—water (30 ml) was added and the mixture washed at approximately 38° C. and the aqueous layer separated;

wash 2—water (25 ml) was added and the mixture washed at approximately 38° C. and the aqueous layer separated;

wash 3—water (25 ml) was added and the mixture washed at approximately 38° C. and the aqueous layer separated.

Water (80 ml) was then added and the mixture heated to 38° C. and sodium hydroxide (47% solution, 6.4 g, 0.076 mol) added to basify the mixture to pH 10-11. The mixture was heated to distil off the DCM in order to afford a solution of Acifluorfen sodium salt. The solution was cooled to room temperature and transferred with the aid of a minimum amount of water to a bottle in order for the solution to be weighed and analysed.

The results for various amounts of acetic anhydride and various reaction temperatures are shown in Table I (see Experiments 1 to 1 1).

### EXAMPLE 2

### General Method for

Nitration of 3-(2-chloro-α,α,α-trifluoro-4-tolyloxy)benzoic acid in Ethylene Dichloride to Yield Acifluorfen

Nitration

Acetic anhydride (see Tables I and II for amounts) was added to 3-(2-chloro-α,α,α-trifluoro-4-tolyloxy)benzoic acid (20 g, 0.063 mol) in ethylene dichloride (54 g, 0.545 mol) and the mixture stirred and heated to 40° C. to dissolve the starting material. The mixture was then cooled to the appropriate reaction temperature (during which time any crystallisation of the starting material was observed). Mixed acid (33.6%, 13 g, 0.069 mol) was added dropwise over a period of 2 hours and the reaction monitored by HPLC for the completion of the reaction. Further additions of Mixed acid were made to reduce the level of starting material to about 1 pph.

Work-Up

The reaction mixture was washed three times as follows:

wash 1—water (30 ml) was added and the mixture washed at approximately 70° C. and the aqueous layer separated;

wash 2—water (25 ml) was added and the mixture washed at approximately 70° C. and the aqueous layer separated;

wash 3—water (25 ml) was added and the mixture washed at approximately 70° C. and the aqueous layer separated.

Water (80 ml) was then added and the mixture heated to 80° C. and sodium hydroxide (47% solution, 6.4 g, 0.076 mol) added to basify the mixture to pH 10-11. The mixture was allowed to separate and the EDC layer was removed. Traces of residual EDC were then removed by distillation to afford a solution of Acifluorfen sodium salt. The solution was cooled to room temperature and transferred with the aid of a minimum amount of water to a bottle in order for the solution to be weighed and analysed.

### EXAMPLE 3

### General Method for

Nitration of 3-(2-chloro-α,α,α-trifluoro-4-tolyloxy)benzoic acid in Perklone to Yield Acifluorfen

The general method and quantities of reagents were exactly as described for Examples 1 and 2 except that the solvent used was Perklone.

The results for Experiments 1 to 45 which were conducted according to the general methods of Examples 1 to 3 are set out in Tables I and II below. In these experiments, the amounts of acetic anhydride, the reaction temperature, the solvent and the quantity of solvent were varied in order to determine the optimum reaction conditions. In each of these experiments, 20 g crude starting material was used containing 84.3% 3-(2-chloro-α,α,α-trifluoro-4-tolyloxy)benzoic acid. In each of the experiments described in Table I, the amount of solvent used was 54.0 g but for the experiments detailed in Table II, the quantity of solvent was varied. In Tables I and II, the term “reactant” refers to 3-(2-chloro-α,α,α-trifluoro-4-tolyloxy) benzoic acid and the following abbreviations are used:

Exp Experiment No.

pph Parts per hundred

Ac2O Acetic anhydride;

DCM Dichloromethane

EDC Ethylene dichloride

TABLE I

| | | | | | | pph | | | | | | | |
|---|---|---|---|---|---|---|---|---|---|---|---|---|---|
| | | Reaction | Ac2O use | HNO3 use | Product | | | | | | Total | | Impurity |
| Exp. | Solvent | Temp ° C. | (mol/mol) | (mol/mol) | Yield % | 2′-nitro | 6′-nitro | Dinitro 1 | Dintro 2 | Dintro 3 | Dinitros | Reactant | Yield % |
| 1 | DCM | −10 | 1.40 | 1.10 | 82.1 | 8.62 | 4.89 | 0.70 | 1.73 | 0.00 | 2.43 | 0.00 | 13.09 |
| 2 | DCM | 0 | 1.40 | 1.10 | 82.4 | 9.39 | 5.56 | 1.52 | 2.12 | 0.53 | 4.17 | 1.30 | 16.83 |
| 3 | DCM | 10 | 1.40 | 1.10 | 85.2 | 10.36 | 6.00 | 0.83 | 2.07 | 0.46 | 3.36 | 0.00 | 16.80 |
| 4 | DCM | −10 | 2.00 | 1.10 | 85.9 | 9.01 | 5.37 | 0.81 | 1.35 | 0.00 | 2.15 | 0.00 | 14.20 |
| 5 | DCM | 0 | 2.00 | 1.10 | 86.1 | 9.58 | 5.79 | 0.81 | 1.77 | 0.39 | 2.96 | 0.00 | 15.78 |
| 6 | DCM | 10 | 2.00 | 1.10 | 84.5 | 10.58 | 6.33 | 0.58 | 0.99 | 0.35 | 1.92 | 0.00 | 15.91 |
| 7 | DCM | −10 | 3.00 | 1.10 | 86.5 | 9.79 | 5.63 | 0.60 | 1.38 | 0.25 | 2.23 | 0.46 | 15.67 |
| 8 | DCM | 0 | 3.00 | 1.10 | 84.3 | 10.56 | 6.17 | 0.52 | 0.90 | 0.00 | 1.42 | 0.00 | 15.30 |
| 9 | DCM | 10 | 3.00 | 1.10 | 83.3 | 11.15 | 6.51 | 0.50 | 0.52 | 0.50 | 1.51 | 1.46 | 17.18 |
| 10 | DCM | 0 | 1.00 | 1.29 | 82.7 | 10.20 | 5.02 | 0.72 | 1.42 | 0.98 | 3.12 | 4.26 | 18.68 |
| 11 | DCM | 0 | 0.50 | 1.42 | 81.7 | 13.23 | 5.48 | 0.84 | 3.67 | 0.71 | 5.23 | 0.00 | 19.57 |
| 12 | EDC | −10 | 1.40 | 1.10 | 86.5 | 8.85 | 4.64 | 0.55 | 1.08 | 0.32 | 1.95 | 1.52 | 14.67 |
| 13 | EDC | 0 | 1.40 | 1.10 | 81.6 | 9.03 | 5.06 | 0.61 | 1.92 | 0.47 | 3.00 | 1.00 | 14.76 |
| 14 | EDC | 10 | 1.40 | 1.10 | 84.6 | 10.21 | 5.45 | 0.93 | 1.74 | 0.54 | 3.21 | 0.00 | 15.96 |
| 15 | EDC | −10 | 2.00 | 1.10 | 84.2 | 8.72 | 4.77 | 0.48 | 0.85 | 0.00 | 1.33 | 0.00 | 12.47 |
| 16 | EDC | 0 | 2.00 | 1.10 | 83.9 | 9.09 | 5.31 | 0.65 | 1.66 | 1.97 | 4.28 | 0.00 | 15.66 |
| 17 | EDC | 10 | 2.00 | 1.10 | 84.2 | 10.21 | 5.90 | 0.44 | 0.81 | 0.49 | 1.74 | 0.00 | 15.04 |
| 18 | EDC | −10 | 3.00 | 1.10 | 85.4 | 9.05 | 4.74 | 0.48 | 0.76 | 0.34 | 1.58 | 1.18 | 14.13 |
| 19 | EDC | 0 | 3.00 | 1.10 | 83.3 | 10.14 | 5.65 | 0.61 | 0.90 | 0.33 | 1.84 | 0.00 | 14.69 |
| 20 | EDC | 10 | 3.00 | 1.10 | 81.6 | 11.12 | 6.21 | 0.48 | 0.25 | 0.52 | 1.25 | 2.08 | 16.86 |
| 21 | EDC | 0 | 1.00 | 1.20 | 80.5 | 9.83 | 4.73 | 0.70 | 1.80 | 1.15 | 3.66 | 5.88 | 19.41 |
| 22 | EDC | 0 | 0.50 | 1.21 | 76.5 | 13.58 | 5.65 | 0.74 | 2.74 | 2.80 | 6.29 | 6.56 | 24.55 |
| 23 | EDC | 10 | AcOH | 1.10 | 56.9 | 15.61 | 6.80 | 1.00 | 1.31 | 0.00 | 2.31 | 43.60 | 38.89 |
| 24 | perklone | −10 | 1.40 | 1.20 | 82.1 | 5.28 | 2.54 | 0.73 | 2.94 | 0.83 | 4.50 | 9.16 | 17.64 |
| 25 | perklone | 0 | 1.40 | 1.16 | 84.7 | 6.36 | 3.06 | 0.56 | 3.43 | 3.61 | 7.60 | 3.39 | 17.29 |
| 26 | perklone | 10 | 1.40 | 1.22 | 82.1 | 7.58 | 3.68 | 0.51 | 3.58 | 1.96 | 6.05 | 2.88 | 16.58 |
| 27 | perklone | −10 | 2.00 | 1.18 | 87.5 | 5.46 | 2.82 | 0.61 | 3.38 | 1.16 | 5.15 | 3.25 | 14.59 |
| 28 | perklone | 0 | 2.00 | 1.20 | 85.3 | 7.03 | 3.61 | 0.59 | 3.44 | 1.96 | 5.98 | 1.86 | 15.76 |
| 29 | perklone | 10 | 2.00 | 1.27 | 84.5 | 7.56 | 3.89 | 0.61 | 3.86 | 2.85 | 7.33 | 1.46 | 17.10 |
| 30 | perklone | −10 | 3.00 | 1.24 | 85.9 | 7.01 | 3.46 | 0.71 | 0.22 | 0.41 | 1.34 | 1.08 | 11.07 |
| 31 | perklone | 0 | 3.00 | 1.21 | 85.9 | 6.29 | 3.66 | 0.66 | 4.49 | 1.84 | 7.00 | 1.07 | 15.47 |
| 32 | perklone | 10 | 3.00 | 1.16 | 82.2 | 8.86 | 4.83 | 0.59 | 1.79 | 1.54 | 3.91 | 0.00 | 14.47 |
| 33 | perklone | 0 | 1.40 | 1.13 | 80.0 | 6.35 | 3.33 | 0.73 | 2.99 | 0.44 | 4.15 | 9.37 | 18.57 |
| 34 | perklone | 10 | 1.40 | 1.13 | 83.0 | 7.80 | 4.02 | 0.70 | 3.55 | 0.75 | 5.01 | 3.67 | 17.01 |
| 35 | perklone | 0-5 | 3.00 | 1.20 | 85.4 | 8.07 | 4.43 | 0.85 | 4.14 | 0.90 | 5.89 | 0.00 | 15.72 |
| 36 | perklone | 0-5 | 3.00 | 1.20 | 84.6 | 7.94 | 4.43 | 0.81 | 3.35 | 1.09 | 5.25 | 0.00 | 14.90 |

TABLE II

| | | Solvent | | | | | pph | | | | | | | |
|---|---|---|---|---|---|---|---|---|---|---|---|---|---|---|
| | | usage | Reaction | Ac₂O use | HNO3 use | Product | 2′- | 6′- | Dinitro | Dintro | Dintro | Total | | Impurity |
| Exp. | Solvent | (g) | Temp ° C. | (mol/mol) | (mol/mol) | Yield % | nitro | nitro | 1 | 2 | 3 | Dinitros | Reactant | Yield % |
| 37 | DCM | 27.0 | −10 | 2.00 | 1.10 | 86.1 | 8.66 | 5.21 | 0.45 | 0.61 | 0.27 | 1.34 | 1.51 | 14.39 |
| 38 | DCM | 54.0 | −10 | 2.00 | 1.10 | 85.9 | 9.01 | 5.37 | 0.81 | 1.35 | 0.00 | 2.15 | 0.00 | 14.20 |
| 38 | DCM | 100.0 | −10 | 2.00 | 1.10 | 83.9 | 9.38 | 5.44 | 0.76 | 1.68 | 0.45 | 2.89 | 0.00 | 14.85 |
| 40 | EDC | 27.0 | −10 | 2.00 | 1.11 | 85.8 | 8.19 | 4.49 | 1.38 | 2.23 | 0.29 | 3.90 | 1.70 | 15.68 |
| 41 | EDC | 54.0 | −10 | 2.00 | 1.10 | 84.2 | 8.72 | 4.77 | 0.48 | 0.85 | 0.00 | 1.33 | 0.00 | 12.47 |
| 42 | EDC | 100.0 | −10 | 2.00 | 1.10 | 83.7 | 9.20 | 4.68 | 0.62 | 1.07 | 0.43 | 2.12 | 0.00 | 13.38 |
| 43 | perklone | 27.0 | −10 | 2.00 | 1.27 | 85.7 | 5.77 | 2.97 | 0.76 | 4.15 | 0.62 | 5.52 | 2.07 | 14.00 |
| 44 | perklone | 54.0 | −10 | 2.00 | 1.18 | 87.5 | 5.46 | 2.82 | 0.61 | 3.38 | 1.16 | 5.15 | 3.25 | 14.59 |
| 45 | perklone | 100.0 | −10 | 2.00 | 1.27 | 84.9 | 5.28 | 2.85 | 0.70 | 4.75 | 0.62 | 6.07 | 2.45 | 14.14 |

The results presented in Table I demonstrate the effects on the concentration of impurities in the final product of changing the molar ratio of acetic anhydride to starting material, temperature and the solvent.

Firstly, the effect of acetic anydride : starting material can be seen from a comparison of the results for Experiments 11, 10, 2, 5 and 8 of Table I, all of which were conducted using DCM as solvent and at a temperature of 0° C. The table shows that while the total concentration of dinitro impurities in the product mixture fell as the ratio of acetic anhydride : starting material increased, the amounts of the 2-nitro and 6-nitro isomers in the product mixture did not follow this pattern. Thus, for acetic anhydride ratios of 0.5, 1.0, 1.4, 2.0 and 3.0, the amounts of 2-nitro isomer present in the product mixture expressed in pph were 13.23, 10.2, 9.39, 9.58 and 10.56 whilst corresponding values for the 6-nitro isomer were 5.48, 5.02, 5.56, 5.79 and 6.17. Since the 2- and 6-nitro isomers are more difficult to separate from Acifluorfen than the dinitro isomers, it is obviously preferable to minimise the production of these mono nitro isomers and, thus, it can be seen that, for optimum performance, the molar ratio of acetic anhydride to starting material must be maintained at from about 1:1 to 3:1.

The effect of temperature can be seen by comparing, for example, the results of Experiments 1 to 3 or 12 to 14 or 24 to 26. It is clear that, in general, the amounts of all the impurities in the product mixture increase as the temperature increases.

Solvent effects are also apparent from Table I and it can be seen that, whilst the amounts of 2-nitro and 6-nitro impurities in the product mixtures are similar for DCM and EDC, they are about 32% lower when Perklone is used as the solvent. Perklone thus appears to be a particularly favourable solvent for use in the present invention.

The results of experiments to test the effect of varying the amount of solvent present in the reaction mixture are shown in Table II. From this table it can be seen that, in general, the amounts of 2-nitro and 6-nitro isomers present in the product mixuture increase as the reaction mixture becomes more dilute.

### EXAMPLE 4

### Nitration of 3-(2-chloro-α,α,α-trifluoro-4-tolyloxy)-N-(methylsulphonyl) Benzamide in Dichloromethane to Yield Acifluorfen

3-(2-chloro-α,α,α-trifluoro-4-tolyloxy)-N-(methylsulphonyl) benzamide (10.4 g, 0.0264 mol) was dispersed in dichloromethane (25.9 g) with stirring. Acetic anhydride (1 1.4 g, 98%, 0.110 mol) was added to the mixture over about 30 minutes maintaining the temperature at about 20° C. Mixed nitric and sulphuric acids (32.6% nitric acid, 0.0317 mol) were added slowly over about 45 minutes, following which the reaction mixture was heated to about 40° to 45° C. for 3 hours. The reaction mass was washed with water and the solvent was removed by distillation to give 10.4 g, 85.2% yield of the required product, Fomesafen. The product mixture also contained 6.8 pph 2-nitro isomer and 5.3 pph 6-nitro isomer.

1. A process for the preparation of a compound of general formula I: 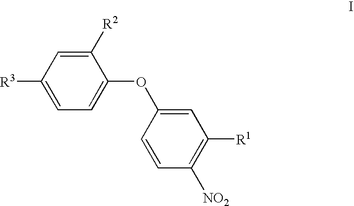
wherein:
R¹ is COOH or CONHSO₂CH₃.;
R² is chloro;
R³ is CF₃;
the process comprising reacting a compound of general formula II: 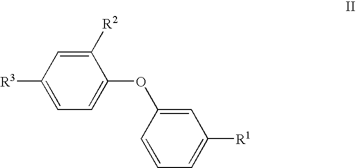
wherein R¹, R² and R³ are as defined for general formula I;
with a nitrating agent comprising nitric acid or a mixture of nitric and sulphuric acids in the presence of an organic solvent and in the presence of acetic anhydride, characterised in that the molar ratio of acetic anhydride to compound of general formula I is from about 1:1 to 3:1 and the organic solvent is tetrachloroethylene (perklone).

2. A process as claimed in claim 1, wherein the weight ratio of solvent to reactant (including any isomers present) is no greater than 4.25:1.

3. A process as claimed in claim 2, wherein the weight ratio of solvent to reactant (including any isomers present) is from 1:1 to 2.5:1.

4. A process as claimed in claim 1 wherein the nitrating agent is a mixture of nitric and sulphuric acids containing from 30 to 45% of pure nitric acid.

5. A process as claimed in claim 1, wherein the nitrating agent is added to the reaction mixture over a period of about 30 minutes to 15 hours.

6. A process as claimed in claim 1, wherein the reaction is carried out at a temperature of −15 to 15° C.

7. A process as claimed in claim 6, wherein the reaction is carried out at a temperature of −10 to 10° C.

8. A process as claimed in claim 1, wherein the compound of general formula I is acifluorfen and which further comprises the steps of converting the acifluorfen to its acid chloride and treating the acid chloride with methane sulphonamide to give fomesafen.
